(19) United States
(12) Patent Application Publication
(10) Pub. No.: US 2004/0138231 A1
(43) Pub. Date: Jul. 15, 2004

(54) Pharmaceutical composition which comprise a solid dispersion of a hydroxypropyl-methylcellulose phthalate polymer

(76) Inventors: Nicola Bateman, Cheshire (GB); Julie Cahill, Cheshire (GB)

Correspondence Address: ROPES & GRAY LLP, ONE INTERNATIONAL PLACE, BOSTON, MA, 02110-2624, US

(21) Appl. No.: 10/468,246

(22) PCT Filed: Feb. 25, 2002
(86) PCT No.: PCT/SE02/00327

(30) Foreign Application Priority Data
Feb. 27, 2001 (GB) 0104752.1

(51) Int. Cl. A61K 31/496; A61K 9/22

(52) U.S. Cl. 514/253.11; 424/468; 514/254.05

(57) ABSTRACT

The invention relates to pharmaceutical compositions, in particular oral pharmaceutical compositions which comprise a solid dispersion of a hydroxypropylmethylcellulose phthalate polymer, preferably HP-55 or HP-55S, and a drug which has pH sensitive solubility.

The invention relates to pharmaceutical compositions, in particular oral pharmaceutical compositions which comprise a solid dispersion of a hydroxypropylmethylcellulose phthalate polymer, preferably HP-55 or HP-55S, and a drug which has pH sensitive solubility.

A common factor which may affect the absorption of a drug when administered orally is the changing pH experienced by the drug as it passes through the gastro-intestinal (GI) tract. Typically a drug may be absorbed in any number of the following sites when administered orally; cheek lining, stomach, duodenum, ileum and colon. The pH may be different at each site of adsorption with the pH significantly different from the stomach (pH 1-3.5) to the small intestine (pH 48). The solubility of the drug may vary with pH leading to the possibility of the drug coming out of solution as it passes through the GI tract. Particular difficulties exist where the drug is dissolved and the solubility decreases in the pH environment found at the site of adsorption. This can possibly lead to low and variable adsorption between doses and different patients.

Various pharmaceutical compounds are adsorbed after administration in the small intestine (in the duodenum, the ileum, or the colon) and have significantly lower solubility in the pH conditions found at the site of adsorption than in the stomach. For such compounds, it would be beneficial to improve the oral bioavailability and/or the variability of adsorption. Such compounds include the following: 1-(6-chloronaphth-2-ylsulfonyl)4[4-(4 pyridyl)benzoyl]piperazine (hereinafter referred to as Compound 1); 1-(5-chloroindol-2-ylsulfonyl)-4-[4-(4-pyridyl)benzoyl]piperazine (hereinafter referred to as Compound 2); 1-(5-chloroindol-2-ylsulfonyl)-4[4-(1-imidazolyl)benzoyl]piperazine (hereinafter referred to as Compound 3); ketoconazole (imidazole antifungal agent); cinnarizine (antihistamine); enoxacin (quinolone antibiotic); cefpodoxime proxetil (oral cephem antibiotic); diazepam; dipyridamole (vasodilator with antithrombotic activity); allopurinol (antigout agent); amiloride hydrochloride (mild diuretic); reserpine (antihypertensive agent). Compounds 1, 2 and 3 are Factor Xa inhibitors and are disclosed in Examples 3 and 6 of WO9957113. Some of the other compounds are discussed in the review article by W Charman et al (Physiochemical and physiological mechanisms for the effects of food on drug absorption: the role of lipids and pH, Journal of Pharmaceutical Sciences, March 1997, Vol 86, No 3, 269-282).

For example we have found that the drug 1-(6-chloronaphth-2-ylsulfonyl)-4-[4-(4 pyridyl)benzoyl]piperazine (Compound 1) is soluble within the acidic pH of the stomach, but is not adsorbed from this area, but has low solubility in the duodenum, ileum and colon which are the main sites of adsorption.

Compound 1 possesses Factor Xa inhibitory activity at concentrations which do not inhibit, or which inhibit to a lesser extent, the enzyme thrombin which is also a member of the blood coagulation enzymatic cascade.

Compound 1 possesses activity in the treatment or prevention of a variety of medical disorders where anticoagulant therapy is indicated, for example in the treatment or prevention of thrombotic conditions such as coronary artery and cerebro-vascular disease. Further examples of such medical disorders include various cardiovascular and cerebrovascular conditions such as myocardial infarction, the formation of atherosclerotic plaques, venous or arterial thrombosis, coagulation syndromes, vascular injury (including reocclusion and restenosis following angioplasty and coronary artery bypass surgery, thrombus formation after the application of blood vessel operative techniques or after general surgery such as hip replacement surgery, the introduction of artificial heart valves or on the recirculation of blood), cerebral infarction, cerebral thrombosis, stroke, cerebral embolism, pulmonary embolism, ischaemia and angina (including unstable angina).

Standard tablet formulations of Compound 1 may not be satisfactory due to the above reasons and have lead to poor oral bioavailability and most importantly high variability in adsorption. Variability is of most concern with any drug affecting the clotting cascade, care is needed since complete blockage of the clotting cascade is an unwanted side effect. On the other hand low exposure levels to the compound will not lead to any therapeutic benefit Therefore, good oral bioavailability is required and, particularly, low variability.

We have found an effective means for providing an oral formulation of drugs whose major site of adsorption is the small intestine, which includes one of the following; the duodenum, the ileum, or the colon, but which have significantly lower solubility in the pH conditions encountered at the site of adsorption than in the stomach, by formulating the drug as a solid dispersion with a hydroxypropylmethylcellulose phthalate polymer (such as HP-50™, HP-55™ or HP-55S™, available from Shin-Etsu Chemical Industry Co., Ltd., Japan or appointed distributors).

In Chem. Pharm. Bull 44(3) 568-571 (1996) a solid dispersion of HP-55 with a poorly water soluble drug is disclosed.

Therefore, we present as a feature of the invention an oral pharmaceutical composition comprising a compound (drug) or a salt thereof which is adsorbed after administration in the small intestine and in which such compound or salt has significantly lower solubility in the pH conditions found at the site of adsorption than in the stomach, in a solid dispersion with a hydroxypropylmethylcellulose phthalate polymer. The hydroxypropylmethylcellulose phthalate polymer is preferably HP-50, HP-55 or HP-55S, or a mixture of any two of these polymers, or a mixture of all three of these polymers. More preferably the polymer is HP-55 or HP-55S or a mixture thereof; most preferably the polymer is HP-55S.

A further feature of the invention is the use of a hydroxypropylmethylcellulose phthalate polymer in improving the oral bioavailabilty and/or variability of adsorption of a compound or a salt thereof which is adsorbed after administration in the small intestine and in which such compound or salt has significantly lower solubility in the pH conditions found at the site of adsorption than in the stomach, by forming a solid dispersion between the polymer and the compound or its salt. The hydroxypropylmethylcellulose phthalate polymer is preferably HP-50, HP-55 or HP-55S, or a mixture of any two of these polymers, or a mixture of all three of these polymers. More preferably the polymer is HP-55 or HP-55S or a mixture thereof; most preferably the polymer is HP-55S.

The use of the term “hydroxypropylmethylcellulose phthalate polymer” is known to the skilled reader for classifying a group of polymers which share the same basic structural features and include such polymers as:

hypromellose phthalate,

methylhydroxypropylcellulosi pthalas,

cellulose, hydrogen 1,2-benzenedicarboxylate, 2-hydroxypropyl methyl,

as well as commercially available polymers HP-55, HP-55S and HP-50.

Preferably the hydroxypropylmethylcellulose phthalate polymer has a Mw of between 20 kDa and 200 kDa., more preferably between 80 kDa and 140 kDa

HP-50, HP-55 and HP-55S are polymers known in the literature and widely used as an enteric coating for oral formulations. HP-55 has a Mw 84 kDa. HP-55S has a Mw of 132 kDa HP-50 has a Mw 78 kDa

By the use of the term “significantly lower solubility in the pH conditions found at the site of adsorption than in the stomach” we mean that the solubility of the compound is at least 10× more soluble in the pH conditions found in the stomach (pH1-2) than the pH conditions found in the small intestine, (pH6-9), preferably 20×, 30×, 40×, 50× and ×100. Compounds having such solubility include 1-(6-chloronaphth-2-ylsulfonyl)-4-[4-(4-pyridyl)benzoyl]piperazine (Compound 1); 1-(5-chloroindol-2-ylsulfonyl)-4-[4-(4-pyridyl)benzoyl]piperazine (Compound 2); 1-(5-chloroindol-2-ylsulfonyl)-4-[4-(1-imidazolyl)benzoyl]piperazine (Compound 3); ketoconazole; cinnarizine; enoxacin; cefpodoxime proxetil; diazepam; dipyridamole; allopurinol; amiloride hydrochloride; reserpine.

We have found the formulation provides significant protection for the compound from the acidic environment of the stomach such that most of the compound is not dissolved. Protecting the compound from the stomach may improve chemical and/or physical stability; for example, it may prevent form changes. When the formulation then reaches the site of adsorption the compound is released, often at an improved maximum supersaturated concentration. For example, when a formulation of Compound 1 reaches the site of adsorption it is released and is able to improve the maximum supersaturated concentration of Compound 1 by 4-6 times.

A preferred ratio of compound to polymer is from 1:10 to 1:0.75; preferably from 1:5 to 1:1.

Preferred compounds are Factor Xa inhibitors, including 1-(6-chloronaphth-2-ylsulfonyl)4[4-(4-pyridyl)benzoyl]piperazine (Compound 1), 1-(5-chloroindol-2-ylsulfonyl)+[4-(4-pyridyl)benzoyl]piperazine (Compound 2) and 1-(5-chloroindol-2-ylsulfonyl)4[4-(1-imidazolyl)benzoyl]piperazine (Compound 3).

The composition may contain from 0.5 mg to 1 g of compound. Additional excipients may be included in the composition.

A further feature of the invention is an oral pharmaceutical composition comprising a compound or a salt thereof which is adsorbed after administration in the small intestine and in which such compound or salt has significantly lower solubility in the pH conditions encountered at the site of adsorption than the stomach, in a solid dispersion with a hydroxypropylmethylcellulose phthalate polymer and one or more fillers, binders, disintegrants or lubricants. The hydroxypropylmethylcellulose phthalate polymer is preferably HP-50, HP-55 or HP-55S, or a mixture of any two of these polymers, or a mixture of all three of these polymers. More preferably the polymer is HP-55 or HP-55S or a mixture thereof; most preferably the polymer is HP-55S.

Suitable fillers include, for example, lactose, sugar, starches, modified starches, mannitol, sorbitol, inorganic salts, cellulose derivatives (e.g. microcrystalline cellulose, cellulose), calcium sulfate, xylitol and lactitol.

Suitable binders include, for example, polyvinylpyrrolidone, lactose, starches, modified starches, sugars, gum acacia, gum tragacanth, guar gum, pectin, wax binders, microcrystalline cellulose, methylcellulose, carboxymethylcellulose, hydroxypropyl methylcellulose, hydroxyethyl cellulose, hydroxypropyl cellulose, copolyvidone, gelatin and sodium alginate.

Suitable disintegrants include, for example, crosscarmellose sodium, crospovidone, polyvinylpyrrolidone, sodium starch glycollate, corn starch, microcrystalline cellulose, hydroxypropyl methylcellulose and hydroxypropyl cellulose.

Suitable lubricants include, for example, magnesium stearate, stearic acid, palmitic acid, calcium stearate, talc, carnuba wax, hydrogenated vegetable oils, mineral oil, polyethylene glycols and sodium stearyl fumarate.

Additional conventional excipients which may be added include preservatives, stabilisers, anti-oxidants, silica flow conditioners, antiadherents or glidants.

Other suitable fillers, binders, disintegrants, lubricants and additional excipients which may be used are described in Handbook of Pharmaceutical Excipients, 3^{rd} Edition, American Pharmaceutical Association, The Theory and Practice of Industrial Pharmacy, 3rd Edition, Lachman, Leon, 1986, Pharmaceutical Dosage Forms: Tablets Volume 1, 2^{nd} Edition, Lieberman, Hebert A., et al, 1989, Modern Pharmaceutics, 3^{rd} Edition Banker, Gilbert and Rhodes, Christopher T, 1995 and Remington's Pharmaceutical Sciences, 20^{th} Edition, 2000.

Typically the compound will be present in an amount-within the range of 1 to 80%, and preferably from 1 to 50% (especially 2 to 15% 2 to 20%) by weight of the composition.

Typically one or more fillers will be present in an amount 1 to 70% by weight.

Typically one or more binders will be present in an amount 2 to 40% by weight.

Typically one or more disintegrants will be present in an amount 1 to 10%, and especially 4 to 6% by weight.

It will be appreciated that a particular excipient may act as both a binder and a filler, or as a binder, a filler and a disintegrant. Typically the combined amount of filler, binder and disintegrant comprises, for example, 1 to 90% by weight of the composition.

Typically one or more lubricants will be present in an amount 0.5 to 3%, and especially 1 to 2% by weight.

Methods for preparing solid dispersions are known in the art and typically comprise the steps of dissolving the compound and the polymer in a common solvent and evaporating the solvent. Methods for evaporating the solvent include rotary evaporation, spray drying, lyophilization and thin film evaporation. Other techniques may be used such as solvent controlled precipitation, pH controlled precipitation, supercritical fluid technology and hot melt extrusion. To aid the process the melt may be extruded with any necessary additional excipient such as a plasticiser, including supercritical fluids

When referring to a solid dispersion we do not exclude the possibility that a proportion of the compound may be dissolved within the polymer used, the exact proportion, if any, will depend upon the physical properties of the compound and the polymer selected.

Preferably 100% of compound in the formulation is in an amorphous form. Whether or not compound (drug) is present in the amorphous form can be determined by conventional thermal analysis. We have found that when solid dispersions of Compound 1 are made then this results in some of Compound 1 being present in the amorphous form, which increases the solubility and dissolution rate of Compound 1. Preferably 100% of Compound 1 in the formulation is in an amorphous form.

The invention is illustrated below by the following non-limiting examples.

### EXAMPLE 1

Preparation of Solid Dispersion

For a 1:5 ratio

0.5 g of drug (Compound 1, hydrochloride salt) and 2.5 g of polymer (HP-55S) are weighed directly into a 250 ml round bottom flask and dissolved in 63 ml of methanol/dichloromethane (50:50). The solvent was removed on the rotary evaporator. The formulation was placed in a vacuum oven and dried under high vacuum at 40° C. for 24 hours.

The formulation was retrieved from the flask and dry milled using the Fritsch mill. The formulation was then dried for a further 24 hours under high vacuum at 40° C.

Weights and volumes for other ratios are pro-rata to the above formulation.

Comparison of Solubility data for Compound 1
and the Antifungal

| Solubility | Antifungal | Compound 1 |
|---|---|---|
| Water | 1.2 ug/ml | <5 ug/ml |
| pH1.2 | 3.6 ug/ml | 250 ug/ml |
| pH6.8 | 1.2 ug/ml | 2 ug/ml |

Antifungal refers to (+)-2-(2,4-difluorophenyl)-3-methyl-1-(1H-1,2,4-triazol-1-yl)-3-[6-(1H-1,2,4-triazol-1-yl)pyridazin-3-ylthio]butan-2-ol (MFB-1041) of which a solid dispersion with HP-55 is disclosed in Kai T., et al. Chem. Pharm. Bull. 44(3) 568-571 (1996).

In Vitro Dissolution of Solid Dispersions

pH Shift Dissolution Method

The formulations were weighed into hard gelatin capsules (equivalent to 25 mg drug) and dissoluted in 500 ml 0.1N HCl for one hour at 37° C. (paddle speed 100 rpm). A 5 ml sample was taken at 55 minutes and the media replaced. After one hour either 10 or 15 mm of a 2.5M KH₂PO₄/16.72% (w/v) NaOH solution was added to the HCl to shift the pH to 6.5 or 7.4 depending on the pH sensitivity of the polymer used in preparation of the solid dispersion. 5 ml samples were then removed with a plastic syringe at 5, 15, 30, 45 and 60 minutes and media replaced after every sampling time point. Each sample was centrifuged (14,000 rpm) at ambient temperature for 15 minutes and then analysed by HPLC using the following conditions:

| | |
|---|---|
| Eluent: | 40% ACN/60% water/0.2% TFA |
| column: | 25 cm HIRPB 4.6 mm i.d.. (with guard) |
| detection wavelength: | 236 nm |
| flow rate: | 1.5 ml/min |
| temperature: | ambient |
| injection volume: | 80 μl |
| retention time: | approximately 6 minutes |

pH 6.5 Dissolution Method

The formulations were weighed into hard gelatin capsules (equivalent to 25 mg drug) and dissoluted in media comprising of 500 ml 0.1N HCl and 110 ml of a 2.5M KH₂PO₄/16.72% (w/v) NaOH solution for one hour at 37° C. (paddle speed 100 rpm). 5 ml samples were then removed with a plastic syringe at 5, 10, 20, 30, 45 and 60 minutes and media replaced after every sampling time point. Each sample was centrifuged (14,000 rpm) at ambient temperature for 15 minutes and then analysed by HPLC using the same conditions as the pH shift method.

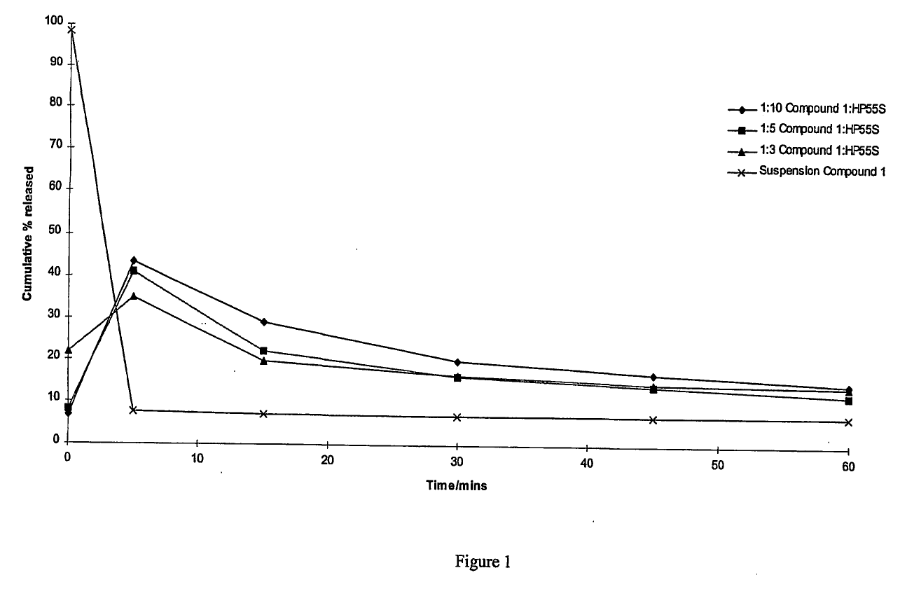

FIG. 1 shows the results of the pH shift in vitro dissolution test performed on solid dispersions made with weight ratios of 1:3, 1:5 and 1:10, Compound 1:HP-55S. A conventional suspension of Compound 1 was included for comparison. All solid dispersion formulations show a significant improvement over the drug in suspension. A reduction in the levels of supersaturation (% released) is seen as the amount of polymer present in the formulation is decreased.

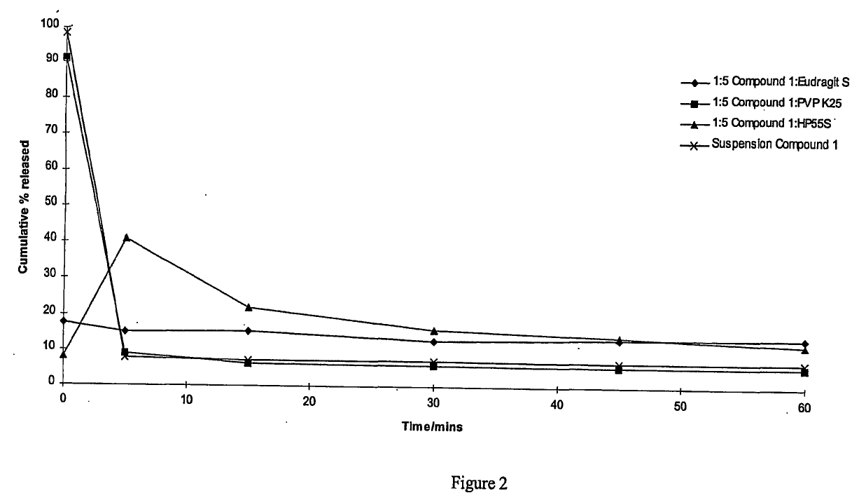

FIG. 2 shows the results of the pH shift in vitro dissolution test performed on the various solid dispersions made with other polymers. This figure demonstrates that the HP-55S polymer is the optimal solid dispersion matrix material since the highest levels of supersaturation are attained with this polymer. The solid dispersions made with PVP do not provide any advantage over a conventional suspension of Compound 1. Similarly to the conventional suspension, on shifting to the higher pH, the PVP formulation is not capable of maintaining supersaturated levels.

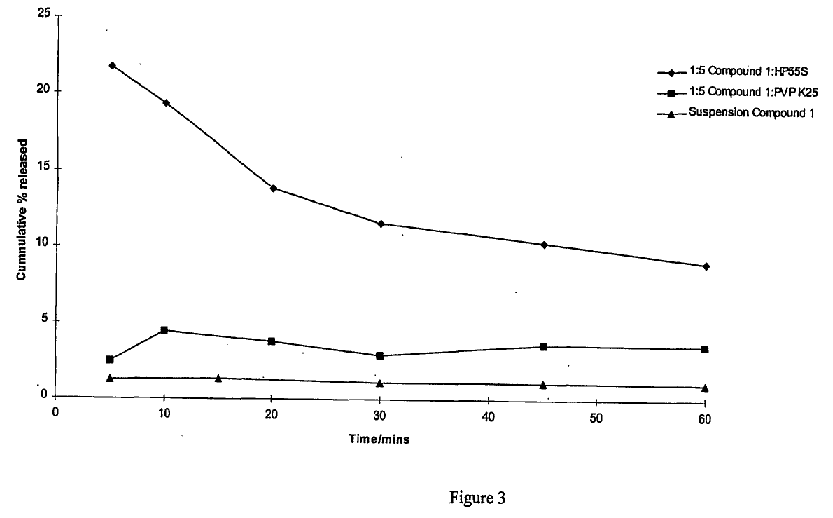

FIG. 3 shows the results of the pH6.5 dissolution test performed on solid dispersions manufactured with PVP and HP-55S. This figure shows that even without prior exposure to acidic media the PVP provides no real enhancement in dissolution over a conventional suspension of Compound 1.

### EXAMPLE 2

Preparation of Solid Dispersion

For a 1:5 ratio

0.5 g of drug (Compound 2, methane sulphonate salt) and 2.5 g of polymer (HP-55S) are weighed directly into a 250 ml round bottom flask and dissolved in 63 ml of methanol/dichloromethane (50:50). The solvent was removed on the rotary evaporator. The formulation was placed in a vacuum oven and dried under high vacuum at 40° C. for 24 hours.

The formulation was retrieved from the flask and dry milled using the Fritsch mill. The formulation was then dried for a further 24 hours under high vacuum at 40° C.

Weights and volumes for other ratios are pro-rata to the above formulation.

Solubility Data for Compound 2

| | Solubility | |
|---|---|---|
| Solvent | (mg/ml) | pH |
| Water | 0.301¹ | 2.6 |
| 0.9% saline | 0.227 | 2.5 |
| 0.1M sodium hydroxide | NR* | 12.7 |
| 0.1M hydrochloric acid | 0.585 | 1.4 |
| pH 3 citrate buffer | 0.193 | 2.9 |
| pH 5 citrate buffer | 0.003 | 5.0 |
| pH 7 phosphate buffer | <0.002 | 7.1 |

Notes on the table above:
¹extremely sensitive on pH, limit detection is 0.0021 mg/ml;
NR* = no result, sample shows evidence of degradation.

In Vitro Dissolution of Solid Dispersions

pH Shift Dissolution Method

The formulations were weighed into hard gelatin capsules (equivalent to 25 mg drug) and dissoluted in 500 ml 0.1N HCl for one hour at 37° C. (paddle speed 100 rpm). A 5 ml sample was taken at 55 minutes and the media replaced. After one hour either 10 or 15 ml of a 2.5M KH₂PO₄/16.72% (w/v) NaOH solution was added to the HCl to shift the pH to 6.5 or 7.4 depending on the pH sensitivity of the polymer used in preparation of the solid dispersion. 5 ml samples were then removed with a plastic syringe at 5, 15, 30, 45 and 60 minutes and media replaced after every sampling time point. Each sample was centrifuged (14,000 rpm) at ambient temperature for 15 minutes and then analysed by HPLC using the following conditions:

| | |
|---|---|
| Eluent: | 50% CAN/50% Water 0.2% TFA |
| Column: | 25 cm × 4.6 mm id HIRPB (with guard) |
| Detection wavelength: | 224 nm |
| Flow rate: | 1.0 ml/min |
| Temperature: | ambient |
| Injection volume: | 80 μl |
| Retention time: | approx 3.7 minutes |

pH 6.5 Dissolution Method

The formulations were weighed into hard gelatin capsules (equivalent to 25 mg drug) and dissoluted in media comprising of 500 nm 0.1N HCl and 10 ml of a 2.5M KH₂PO₄/16.72% (w/v) NaOH solution for one hour at 37° C. (paddle speed 100 rpm). 5 ml samples were then removed with a plastic syringe at 5, 10, 20, 30, 45 and 60 minutes and media replaced after every sampling time point. Each sample was centrifuged (14,000 rpm) at ambient temperature for 15 minutes and then analysed by HPLC using the same conditions as the pH shift method.

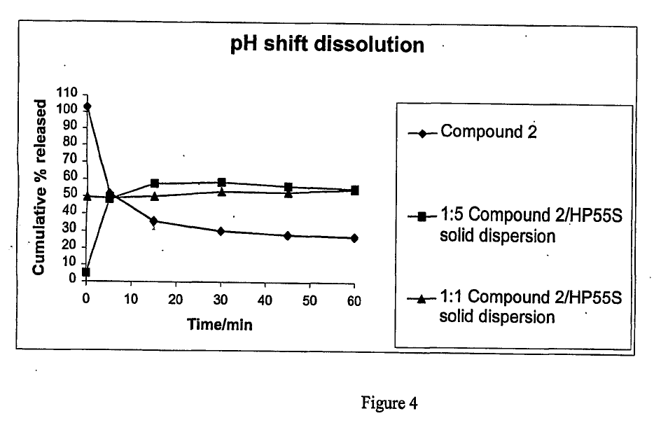

FIG. 4 shows the results of the pH shift in vitro dissolution test performed on solid dispersions made with weight ratios of 1:1 and 1:5, Compound 2:HP-55S. A conventional suspension of Compound 2 was included for comparison. All solid dispersion formulations, show a significant improvement over the drug in suspension. No overall reduction in the levels of super saturation (% released) is seen as the amount of polymer present in the formulation is decreased.

1. An oral pharmaceutical composition comprising a compound or a salt thereof which is adsorbed after administration in the small intestine and in which such compound or salt has significantly lower solubility in the pH conditions found at the site of adsorption than in the stomach, in a solid dispersion with a hydroxypropylmethylcellulose phthalate polymer.

2. An oral pharmaceutical composition as claimed in claim 1 in which the polymer is HP-50, HP-55, HP-55S or a mixture thereof.

3. An oral pharmaceutical composition as claimed in claim 1 or 2 which additionally comprises one or more fillers, binders, disintegrants or lubricants.

4. An oral pharmaceutical composition as claimed in any one of claims 1 to 3 wherein the ratio of compound to polymer is from 1:10 to 1:0.75.

5. An oral pharmaceutical composition as claimed in claim 4 wherein the ratio of compound to polymer is from 1:5 to 1:1.

6. An oral pharmaceutical composition as claimed in any of claims 1 to 5 wherein the composition comprises from 0.5 mg to 1 g of compound.

7. An oral pharmaceutical composition as claimed in any of claims 1 to 6 wherein the compound is a Factor Xa inhibitor.

8. An oral pharmaceutical composition as claimed in claim 7 wherein the compound is selected from 1-(6-chloronaphth-2-ylsulfonyl)-4-[4-(4-pyridyl)benzoyl]piperazine, 1-(5-chloroindol-2-ylsulfonyl)4[4-(4-pyridyl)benzoyl]piperazine and 1-(5-chloroindol-2-ylsulfonyl)4[4-(1-imidazolyl)benzoyl]piperazine.

9. Use of a hydroxypropylmethylcellulose phthalate polymer in improving the oral bioavailabilty and/or variability of adsorption of a compound or a salt thereof which is adsorbed after administration in the small intestine and in which such compound or salt has significantly lower solubility in the pH conditions found at the site of adsorption than in the stomach, by forming a solid dispersion between the polymer and the compound or its salt.

10. Use as claimed in claim 9 in which the polymer is HP-50, HP-55, HP-55S or a mixture thereof.
